(19) United States
(12) Patent Application Publication
(10) Pub. No.: US 2017/0202464 A1
(43) Pub. Date: Jul. 20, 2017

(54) PULSE WAVE VELOCITY METER

(71) Applicants: LITE-ON ELECTRONICS (GUANGZHOU) LIMITED, GUANGZHOU (CN); LITE-ON TECHNOLOGY CORPORATION, Taipei (TW)

(72) Inventor: Tzu-Hao Tsao, Taipei (TW)

(21) Appl. No.: 15/333,293

(22) Filed: Oct. 25, 2016

Related U.S. Application Data
(60) Provisional application No. 62/278,996, filed on Jan. 15, 2016.

(30) Foreign Application Priority Data
May 27, 2016 (CN) 201610369966.1

(51) Int. Cl. A61B 5/021; A61B 5/00; A61B 5/024; A61B 5/0408

(52) U.S. Cl. CPC A61B 5/02125; A61B 5/04085; A61B 5/6893; A61B 5/6897; A61B 5/6898; A61B 5/02416

(57) ABSTRACT

A pulse wave velocity meter includes a carrier, a photo-plethysmography (PPG) sensor, an electrocardiography (ECG) right-hand electrode, an ECG left-hand electrode, a PPG sensing unit, an ECG sensing unit and a processing unit. The PPG sensing unit is electrically connected to the PPG sensor. The ECG sensing unit is electrically connected to the ECG right-hand electrode and the ECG left-hand electrode. The processing unit is electrically connected to the PPG sensing unit and the ECG sensing unit. The PPG sensor, the ECG right-hand electrode and the ECG left-hand electrode respectively are disposed at a plurality of separation regions of the carrier.

This application claims the benefit of U.S. provisional application Ser. No. 62/278,996, filed Jan. 15, 2016, the subject matter of which is incorporated herein by reference, and claims the benefit of People's Republic of China application Serial No. 201610369966.1, filed May 27, 2016, the subject matter of which is incorporated herein by reference.

### BACKGROUND OF THE INVENTION

Field of the Invention

The invention relates in general to a pulse wave velocity meter, and more particularly to a pulse wave velocity meter having multiple electrodes.

Description of the Related Art

The conventional pulse wave velocity meter includes a photo-plethysmography (PPG) sensor, an electrocardiography (ECG) left-hand electrode and an ECG right-hand electrode. Human body's physiological information can be obtained when a user contacts the PPG sensor, the ECG left-hand electrode and the ECG right-hand electrode using his/her finger. However, if the user's finger has poor contact with the PPG sensor and the ECG electrode, the obtained PPG signal and ECG signal will contain many noises, and the quality of the obtained physiological information will deteriorate.

Therefore, how to improve the contact quality between the finger and the PPG sensor and the ECG electrode has become a prominent task for the industries of the technology field.

### SUMMARY OF THE INVENTION

The invention provides a pulse wave velocity meter capable of resolving the above problems encountered in the prior art.

According to one embodiment of the invention, a pulse wave velocity meter is provided. The pulse wave velocity meter includes a carrier, a photo-plethysmography (PPG) sensor, an electrocardiography (ECG) right-hand electrode, an ECG left-hand electrode, a PPG sensing unit, an ECG sensing unit and a processing unit. The PPG sensing unit is electrically connected to the PPG sensor. The ECG sensing unit is electrically connected to the ECG right-hand electrode and the ECG left-hand electrode. The processing unit is electrically connected to the PPG sensing unit and the ECG sensing unit. The PPG sensor, the ECG right-hand electrode and the ECG left-hand electrode respectively are disposed at a plurality of separation regions of the carrier.

According to another embodiment of the invention, the said pulse wave velocity meter further includes a ground/ right leg drive electrode. The PPG sensor, the ECG right-hand electrode, the ground/RLD electrode and the ECG left-hand electrode respectively are disposed at a plurality of separation regions of the carrier.

According to another embodiment of the invention, the carrier of the said pulse wave velocity meter includes a front surface and a rear surface disposed oppositely. The PPG sensor and the ground/RLD electrode are disposed at two separation regions on the front surface. The ECG right-hand electrode and the ECG left-hand electrode are disposed at two separation regions on the rear surface. The PPG sensor is opposite to one of the ECG right-hand electrode and the ECG left-hand electrode. The ground/RLD electrode is opposite to the other one of the ECG right-hand electrode and the ECG left-hand electrode.

According to another embodiment of the invention, the said pulse wave velocity meter is a communication device. The carrier is at least a portion of a casing of the communication device. The casing includes a front surface and a rear surface disposed oppositely. The PPG sensor is disposed on the front surface. The ground/RLD electrode, the ECG right-hand electrode and the ECG left-hand electrode are disposed on the rear surface. The ECG right-hand electrode and the ground/RLD electrode are adjacent to an edge of the carrier. The ECG left-hand electrode is adjacent to another edge of the carrier.

According to another embodiment of the invention, the said pulse wave velocity meter is a communication device. The carrier is at least a portion of a casing of the communication device. The casing includes a front surface and a rear surface disposed oppositely. The PPG sensor is disposed on the front surface. The ground/RLD electrode, the ECG right-hand electrode and the EGG left-hand electrode are disposed on the rear surface. The ECG right-hand electrode and the ground/RLD electrode are adjacent to an edge of the casing. The ECG left-hand electrode is adjacent to another edge of the casing.

According to another embodiment of the invention, the said pulse wave velocity meter is a communication device. The carrier is at least a portion of a casing of the communication device. The casing includes a front surface and a plurality of lateral surfaces disposed oppositely. The PPG sensor, the ground/RLD electrode and the ECG left-hand electrode are disposed on the lateral surfaces. The ECG right-hand electrode is disposed on the front surface.

According to another embodiment of the invention, the said pulse wave velocity meter is a steering wheel of a vehicle. The carrier is a handle of the steering wheel. The handle includes a front surface. The ECG left-hand electrode and the ground/RLD electrode are disposed on the left side of the front surface. The PPG sensor and the ECG right-hand electrode are disposed on the right side of the front surface.

According to another embodiment of the invention, the said pulse wave velocity meter is a steering wheel of a vehicle. The carrier is a handle of the steering wheel. The handle includes a rear surface. The PPG sensor and the ECG right-hand electrode are disposed on the right side of the rear surface. The ECG left-hand electrode and the ground/RLD electrode are disposed on the left side of the rear surface.

According to another embodiment of the invention, the said pulse wave velocity meter is a steering wheel of a vehicle. The carrier is a handle of the steering wheel. The handle includes a front surface and a rear surface. The PPG sensor is disposed on the right side of the front surface. The ECG right-hand electrode is disposed on the right side of the rear surface. The ECG left-hand electrode is disposed on the left side of the front surface. The ground/RLD electrode is disposed on the left side of the rear surface.

According to another embodiment of the invention, the carrier of the said pulse wave velocity meter has a second recess portion at which the ground/RLD electrode is disposed.

According to another embodiment of the invention, the said pulse wave velocity meter further includes a protrusion portion disposed on the ground/right leg drive (RLD) electrode.

According to another embodiment of the invention, the carrier of the said pulse wave velocity meter has a first recess portion at which the PPG sensor is disposed.

According to another embodiment of the invention, the said pulse wave velocity meter further includes a first convex ring disposed on the ECG right-hand electrode.

According to another embodiment of the invention, the said pulse wave velocity meter further includes a second convex ring disposed on the ECG left-hand electrode.

According to another embodiment of the invention, the carrier of the said pulse wave velocity meter is a casing of an electronic device.

According to another embodiment of the invention, the carrier of the said pulse wave velocity meter is a handle of a steering wheel.

According to another embodiment of the invention, the said pulse wave velocity meter is a keypad, a communication device or a steering wheel.

The above and other aspects of the invention will become better understood with regard to the following detailed description of the preferred but non-limiting embodiment(s). The following description is made with reference to the accompanying drawings.

### BRIEF DESCRIPTION OF THE DRAWINGS

FIG. 1 is a front view of a pulse wave velocity meter according to a first embodiment of the invention.

FIG. 2 is a back view of the pulse wave velocity meter of FIG. 1.

FIGS. 3-5 are schematic diagrams of the pulse wave velocity meter of FIGS. 1-2 for measuring human body's physiological information.

FIG. 6 is a front view of a pulse wave velocity meter according to a second embodiment of the invention.

FIG. 7 is a back view of the pulse wave velocity meter of FIG. 6.

FIG. 8 is a front view of a pulse wave velocity meter according another embodiment of the invention.

FIG. 9 is a front view of a pulse wave velocity meter according to other embodiment of the invention.

FIG. 10 is a back view of he pulse wave velocity meter of FIG. 9.

FIG. 11 is a front view of a pulse wave velocity meter according to a third embodiment of the invention.

FIG. 12A is a front view of a pulse wave velocity meter according to a fourth embodiment of the invention.

FIG. 12B is a back view of the pulse wave velocity meter of FIG. 12A.

FIG. 12C is a partial cross-sectional view of the pulse wave velocity meter of FIG. 12A viewed along a direction 12C-12C′.

### DETAILED DESCRIPTION OF THE INVENTION

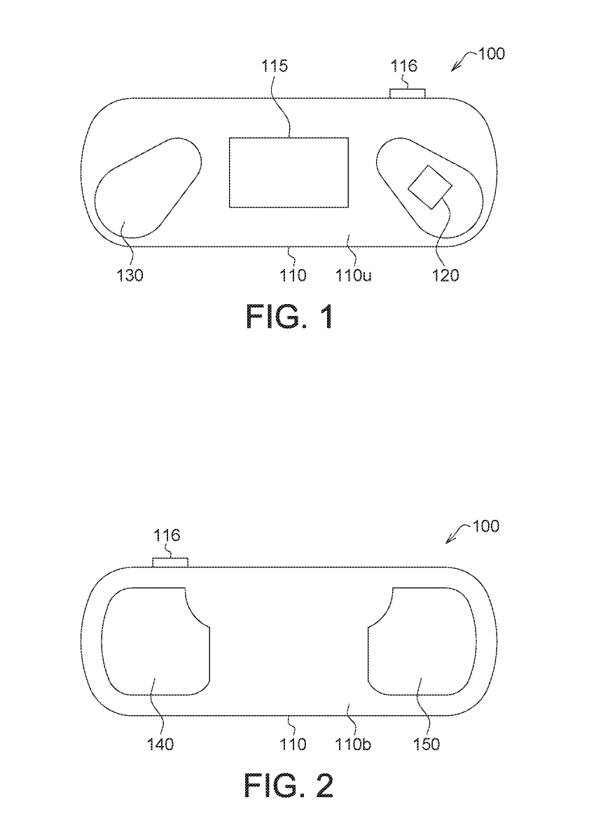

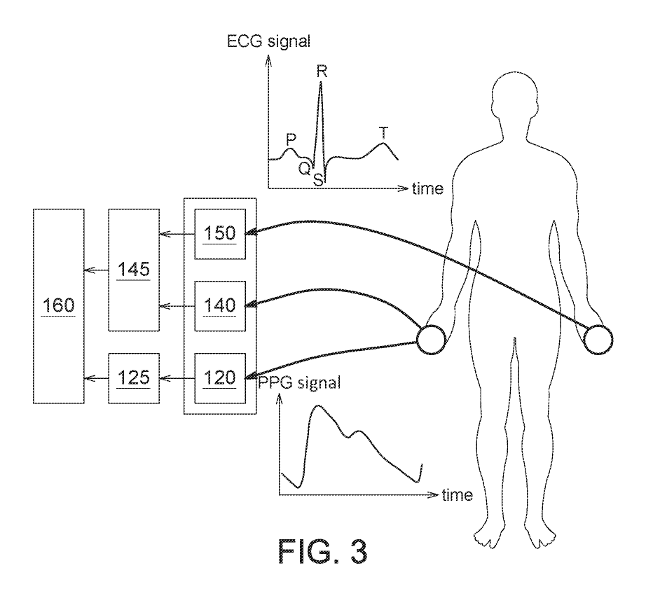

Refer o FIG. 1 to FIG. 3. FIG. 1 is a front view of a pulse wave velocity meter according to a first embodiment of the invention. FIG. 2 is a back view of the pulse wave velocity meter of FIG. 1. FIG. 3 is a schematic diagram of the pulse wave velocity meter of FIGS. 1-2 for measuring human body's physiological information.

The pulse wave velocity meter 100 includes a carrier 110, a display unit 115, a power key 116, a photo-plethysmography (PPG) sensor 120, a PPG sensing unit 125, a ground/right leg drive (ground/RLD) electrode 130, an electrocardiography (ECG) right-hand electrode 140, an ECG sensing unit 145, an ECG left-hand electrode 150 and a processing unit 160. The ground/RLD electrode 130 provides a signal or mechanism for eliminating or reducing the noises.

The pulse wave velocity meter 100 can be turned on or turned off by pressing the power key 116. The carrier 110 is, for example, at least a portion of a casing of the pulse wave velocity meter 100.

The display unit 115, the power key 116, the PPG sensor 120, the ground/RLD electrode 130, the ECG right-hand electrode 140 and the ECG left-hand electrode 150 are disposed inside the carrier 110. The PPG sensing unit 125, the ECG sensing unit 145 and the processing unit 160 are disposed inside the carrier 110, for example, on a circuit board (not illustrated) inside the carrier 110. The PPG sensor 120, the ground/RLD electrode 130, the ECG right-hand electrode 140 and the ECG left-hand electrode 150 are exposed outside the carrier 110.

Additionally, the display unit 115, the power key 116, the PPG sensor 120, the PPG sensing unit 125, the ground/RLD electrode 130, the ECG right-hand electrode 140, the ECG sensing unit 145, the ECG left-hand electrode 150 are electrically connected to the processing unit 160. The 2.0 connection relationship between the sensing unit, the sensor, the electrode and the processing unit is further elaborated below. The PPG sensing unit 125 is electrically connected to the PPG sensor 120. The ECG sensing unit 145 is electrically connected to the ECG right-hand electrode 140 and the ECG left-hand electrode 150. The processing unit 160 is electrically connected to the PPG sensing unit 125 and the ECG sensing unit 145.

The PPG sensor 120, the ground/RLD electrode 130, the ECG right-hand electrode 140 and the ECG left-hand electrode 150 respectively are disposed at a plurality of separation regions of the carrier 110. That is, one of the coverage area of the PPG sensor 120, the coverage area of the ground/RLD electrode 130, the coverage area of the ECG right-hand electrode 140 and the coverage area of the ECG left-hand electrode 150 does not overlap or connect with any other coverage area on the same side or the same surface of the carrier 110, so that the user does not need to contact two or more than two sensors or electrodes using the same finger at the same time.

The ECG right-hand electrode 140 and the EGG left-hand electrode 150 respectively are disposed at two separation regions, and therefore are capable of providing the user with a large and sufficient contact region, for example, basically equivalent to or larger than the area of the contact surface of the finger. In an embodiment, the area of the ECG right-hand electrode 140 is substantially equivalent to that of the ECG left-hand electrode 150. However, the present invention does not restrict the relationship between the area of the ECG right-hand electrode 140 and that of the ECG left-hand electrode 150, and the area of the ECG right-hand electrode 140 can be larger than, equivalent to or smaller than that of the ECG left-hand electrode 150.

As indicated in FIGS. 1 and 2, the carrier 110 has a front surface 110u and a rear surface 110b disposed oppositely. In the present embodiment, the PPG sensor 120 and the ground/RLD electrode 130 are disposed at two separation regions on the front surface 110u. The EGG right-hand electrode 140 and the EGG left-hand electrode 150 are disposed at two separation regions on the rear surface 110b.

Referring to FIGS. 3-5, schematic diagrams of the pulse wave velocity meter of FIGS. 1-2 for measuring human body's physiological information are shown. The physiological information includes, for example, pulse wave velocity (PWV), blood pressure, heart rate, body fat, blood glucose, pulse oximetry and other physiological information. The measured physiological information can be displayed on the display unit 115 (illustrated in FIG. 1).

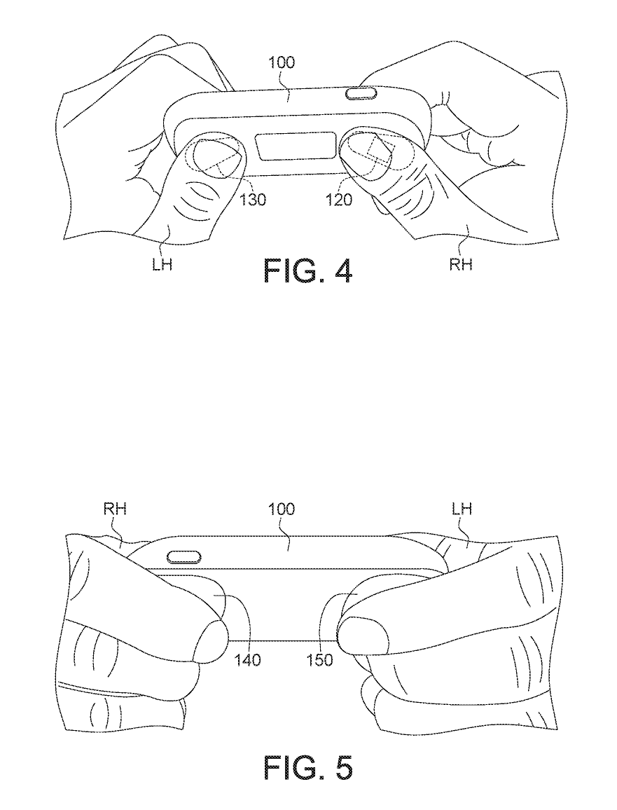

As indicated in FIGS. 4 and 5, a finger, such as the left-hand (LH) thumb can contact the ground/RLD electrode 130, and another finger, such as the LH index finger or middle finger can contact the ECG left-hand electrode 150. Furthermore, a finger, such as the right-hand (RH) thumb can contact the PPG sensor 120, and another finger, such as the RH index finger or middle finger can contact the ECG right-hand electrode 140. Thus, the ECG signal and the PPG signal of the human body can be obtained.

In another embodiment, as long as the ECG signal belonging to the first ECG lead I can be obtained, that which contacts the ECG right-hand electrode 140 and the ECG left-hand electrode 150 does not necessarily have to be a human finger. For example, the user can obtain the ECG signal belonging to the first ECG lead I by contacting the ECG right-hand electrode 140 and the ECG left-hand electrode 150 using a body part between his/her elbow and fingers.

As indicated in FIG. 3, the ECG signal can be transmitted to the ECG sensing unit 145 through the ECG right-hand electrode 140 and the EGG left-hand electrode 150. The ECG sensing unit 145 can convert the voltage potential difference between the ECG right-hand electrode 140 and the ECG left-hand electrode 150 into an electrical signal for the processing unit 160 to process. The PPG signal can be transmitted to the PPG sensing unit 125 through the PPG sensor 120. The PPG sensing unit 125 can convert the PPG signal, being a light signal, into an electrical signal for the processing unit 160 to process.

The electrical signal can be transmitted to the processing unit 160 through PPG sensing unit 125 and the ECG sensing unit 145. The processing unit 160 can analyze or process the electrical signal to obtain human body's physiological information, such as pulse wave velocity, blood pressure, heart rate, body fat, blood glucose, pulse oximetry and other the physiological information. The pulse wave velocity and the blood pressure can be obtained from the ECG signal and the PPG signal. The heart rate can be obtained from the ECG signal or the PPG signal. The pulse oximetry can be obtained from the PPG signal.

Refer to FIG. 1 and FIG. 2. The PPG sensor 120, the ground/RLD electrode 130, the ECG right-hand electrode 140 and the ECG left-hand electrode 150 respectively are disposed at a plurality of separation regions of the carrier 110 of a casing, and therefore are capable of providing the user with a large and sufficient contact region to improve the quality of electrical contact between the user's finger (or other body parts) and the electrode and the sensor. Therefore, the noises contained in the PPG and the ECG signal can be reduced to obtain more accurate PPG and ECG signals and increase the accuracy of the physiological information.

In other embodiment, the PPG sensor 120, the ground/RLD electrode 130, the ECG right-hand electrode 140, the ECG left-hand electrode 150, the PPG sensing unit 125, the ECG sensing unit 145 arid the processing unit 160 can be integrated into other device or environment, such as keypad, desk, chair, toilet, bathroom, appliance, transportation (such as vehicle, motor cycle or bicycle), in which the pulse wave velocity meter 100 can be disposed or installed.

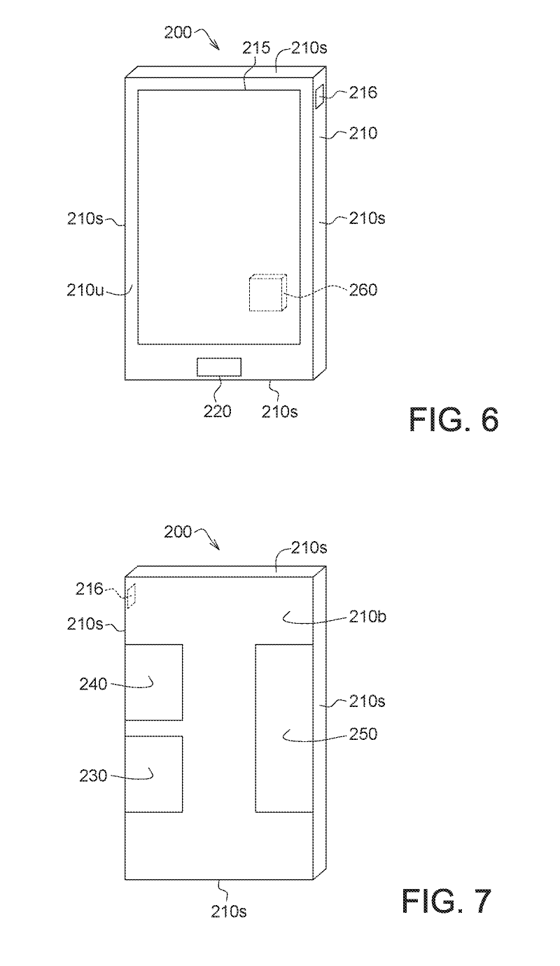

Refer to FIGS. 6 and 7. FIG. 6 is a front view of a pulse wave velocity meter 200 according to a second embodiment of the invention. FIG. 7 is a back view of the pulse wave velocity meter 200 of FIG. 6.

In the present embodiment, the pulse wave velocity meter 200 is a communication device, such as a mobile phone. Apart from providing a physiological information measuring function, the pulse wave velocity meter 200 further provides necessary functions of an ordinary communication device.

The pulse wave velocity meter 200 at least includes a carrier 210, a display unit 215, a power key 216, a PPG sensor 220, a PPG sensing unit (not illustrated), a ground/RLD electrode 230, an ECG right-hand electrode 240, an ECG sensing unit (not illustrated), an ECG left-hand electrode 250 and a processing unit 260. The carrier 210 is, for example, at least a portion of a casing of the pulse wave velocity meter 200.

The display unit 215, the power key 216, the PPG sensor 220, the ground/RLD electrode 230, the ECG right-hand electrode 240 and the ECG left-hand electrode 250 are disposed on the carrier 210. The PPG sensing unit, the ECG sensing unit and the processing unit 260 are disposed inside the carrier 210, for example, on a circuit board (not illustrated) inside the carrier 210. The PPG sensor 220, the ground/RLD electrode 230, the ECG right-hand electrode 240 and the ECG left-hand electrode 250 can be exposed outside the carrier 210.

Besides, the display unit 215, the power key 216, the PPG sensor 220, the PPG sensing unit, the ground/RLD electrode 230, the ECG right-hand electrode 240, the ECG sensing unit and the ECG left-hand electrode 250 are electrically connected to the processing unit 260. The relationship of electrical connection between the sensing unit, the sensor, electrode and the processing unit are the same as the connection relationship between the elements of the pulse wave velocity meter 100 of the first embodiment, and the similarities are not repeated here.

The PPG sensor 220, the ground/RLD electrode 230, the ECG right-hand electrode 240 and the EGG left-hand electrode 250 are disposed at a plurality of separation regions of the carrier 210. The carrier 210 has a front surface 210u and a rear surface 210b disposed oppositely. In the present embodiment, the PPG sensor 220 is disposed on the front surface 210u. The ground/RLD electrode 230, the ECG right-hand electrode 240 and the ECG left-hand electrode 250 are disposed on the rear surface 210b. If the ground/RLD electrode 230 and the ECG right-hand electrode 240 are adjacent to an edge (such as a long side) of the carrier 210, then the ECG left-hand electrode 250 can be adjacent to another edge (such as another long side) of the carrier 210 to match the user's gripping habit. In another embodiment, at least one of the PPG sensor 220, the ground/RLD electrode 230, the ECG right-hand electrode 240 and the ECG left-hand electrode 250 is disposed on the front surface 210u, and the others can be disposed on the rear surface 210b. Or, at least one of the PPG sensor 220, the ground/RLD electrode 230, the ECG right-hand electrode 240 and the ECG left-hand electrode 250 is disposed on one of the front surface 210u, the rear surface 210b and the lateral surface 210s of the carrier 210, and the others of the PPG sensor 220, the ground/RLD electrode 230, the ECG right-hand electrode 240 and the ECG left-hand electrode 250 can be disposed on another one of the front surface 210u, the rear surface 210b and the lateral surface 210s.

Although it is not illustrated in FIG. 6 and FIG. 7, the user can contact the ECG left-hand electrode 250 using one left-hand finger, contact the ground/RLD electrode 230 using another left-hand finger, contact the PPG sensor 220 using one right-hand finger, and contact the ECG right-hand electrode 240 using another right-hand finger to obtain the ECG signal and the PPG signal of the human body.

As disclosed above, at least one of the PPG sensor, the ground/RLD electrode, the ECG right-hand electrode and the ECG left-hand electrode can be disposed on the front surface of the carrier facing towards the user, and another one or some of the PPG sensor, the ground/RLD electrode, the ECG right-hand electrode and the ECG left-hand electrode can be disposed on the rear surface or a lateral surface of the carrier. In the embodiments of the invention, any disposition of the electrodes and the sensors will do as long as the sensing region of each electrode or sensor is separated to contact the human skin by a maximum area.

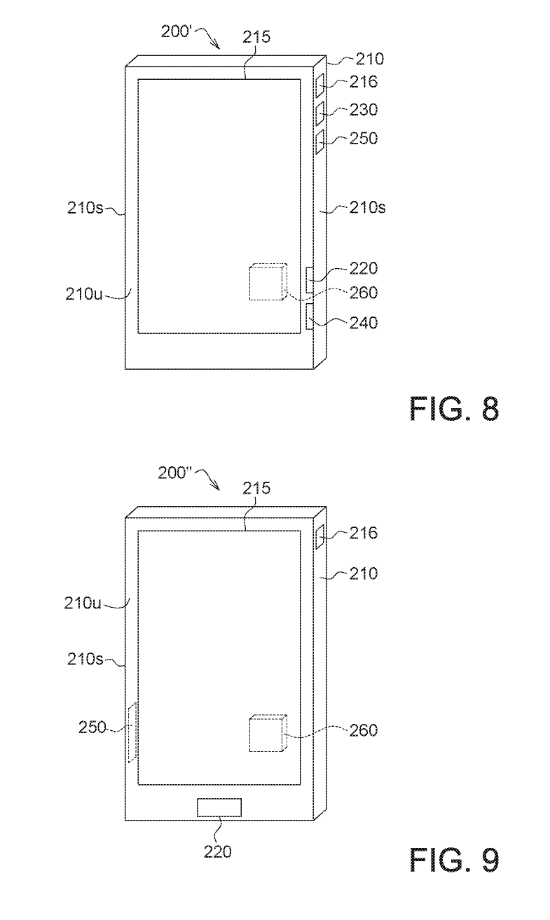

Referring to FIG. 8, a front view of a pulse wave velocity meter 200′ according another embodiment of the invention is shown. As indicated in FIG. 8, the ground/RLD electrode 230 and the ECG left-hand electrode 250 are disposed on the lateral surface 210s at the right side of the carrier 210, and the ECG right-hand electrode 240 and the PPG sensor 220 are disposed at the right side the front surface 210u of the carrier 210 to match left-handed users' gripping habit. Under such design, when a user holds the carrier 210 with his/her left hand, the user can contact the ground/RLD electrode 230 and the ECG left-hand electrode 250 using his/her left-hand index finger and middle finger respectively and contact the ECG right-hand electrode 240 and the PPG sensor 220 using his/her right-hand index finger and middle finger respectively to obtain the ECG signal and the PPG signal of the human body. In another embodiment, the ECG right-hand electrode 240 and the PPG sensor 220 can be disposed at the left side of the front surface 210u of the carrier 210.

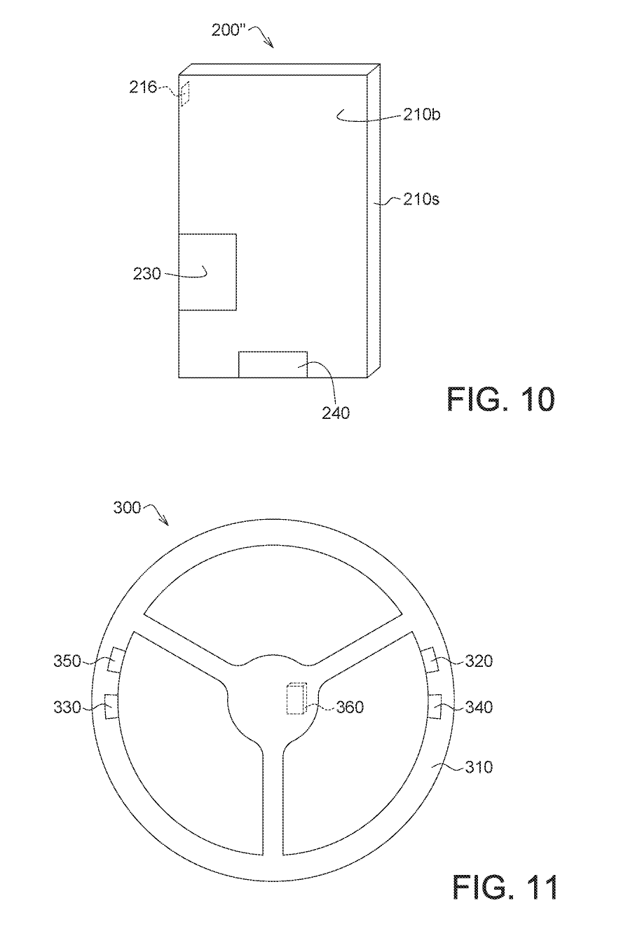

Refer to FIGS. 9 and 10. FIG. 9 is a front view of a pulse wave velocity meter 200″ according to other embodiment of the invention. FIG. 10 is a back view of the pulse wave velocity meter 200″ of FIG. 9. The PPG sensor 220 is disposed on the front surface 210u of the carrier 210. The ECG left-hand electrode 250 is disposed on the lateral surface 210s at the left side of the carrier 210. The ECG right-hand electrode 240 and the ground/RLD electrode 230 are disposed on the rear surface 210b of the carrier 210. The ECG right-hand electrode 240 can be opposite to the PPG sensor 220. Under such design, when a user holds the carrier 210 using his/her left hand, the user can contact the ECG left-hand electrode 250 using his/her left-hand thumb, contact the ground/RLD electrode 230 using his/her left-hand index finger or middle finger, contact the PPG sensor 220 using his/her right-hand thumb, and contact the ECG right-hand electrode 240 using his/her right-hand index finger or middle finger to obtain the ECG signal and the PPG signal of the human body.

Referring to FIG. 11, a front view of a pulse wave velocity meter 300 according to a third embodiment of the invention is shown.

In the present embodiment, the pulse wave velocity meter 300 is such as the steering wheel of a vehicle. Apart from providing a physiological information measuring function, the pulse wave velocity meter 300 further controls the travelling direction of the vehicle.

The pulse wave velocity meter 300 at least includes, for example, a display unit (not illustrated) of a vehicle screen, a PPG sensing unit (not illustrated), an ECG sensing unit (not illustrated), a carrier 310 (such as the handle of the steering wheel), a PPG sensor 320, a ground/RLD electrode 330, an ECG right-hand electrode 340, an ECG left-hand electrode 350 and a processing unit 360.

The PPG sensor 320, the ground/RLD electrode 330, the ECG right-hand electrode 340 and the ECG left-hand electrode 350 are disposed on the handle of the steering wheel. The PPG sensor 320, the ground/RLD electrode 330, the ECG right-hand electrode 340 and the ECG left-hand electrode 350 can be exposed outside the handle of the steering wheel. The PPG sensing unit, the ECG sensing unit and the processing unit 360 are disposed inside the vehicle body of the vehicle, for example, on a circuit board (not illustrated) inside the vehicle body.

Moreover, the vehicle screen, the PPG sensor 320, the PPG sensing unit, the ground/RLD electrode 330, the ECG right-hand electrode 340, the ECG sensing unit and the ECG left-hand electrode 350 are electrically connected to the processing unit 360. The PPG sensor 320, the ground/RLD electrode 330, the ECG right-hand electrode 340 and the ECG left-hand electrode 350 are disposed at a plurality of separation regions of a handle of a steering wheel. In the present embodiment, the PPG sensor 320 and the ECG right-hand electrode 340 are disposed at the right side of the front surface of the carrier 310, that is, the right side of the surface of the carrier 310 facing the user. The ECG left-hand electrode 350 and the ground/RLD electrode 330 are disposed at the left side of the front surface. Under such design, when a user holds the handle of the steering wheel using his/her two hands, the user can contact the ground/RLD electrode 330 using his/her left-hand thumb, contact the ECG left-hand electrode 350 using his/her left-hand index finger, contact the PPG sensor 320 using his/her right-hand index finger, and contact the ECG right-hand electrode 340 using his/her right-hand thumb to obtain the ECG signal and the PPG signal of the human body.

In another embodiment, the PPG sensor 320 and the ECG right-hand electrode 340 are disposed at the right side of the rear surface (opposite to the front surface) of the carrier 310 (the handle) of FIG. 11. The ECG left-hand electrode 350 and the ground/RLD electrode 330 are disposed at the left side of the rear surface. The user can contact the PPG sensor 320 and the ECG right-hand electrode 340 using his/her right-hand index finger and middle finger respectively, and contact the ECG left-hand electrode 350 and the ground/RLD electrode 330 using his/her left-hand index finger and middle finger respectively. In other embodiment as indicated in FIG. 11, the positions of the ECG left-hand electrode 350 and the PPG sensor 320 remain the same, but the ground/RLD electrode 330 and the ECG right-hand electrode 340 can change to be disposed at the positions opposite to that illustrated in the diagram. Under such design, when a user holds the carrier 310, the user can contact the ECG left-hand electrode 350 using his/her left-hand thumb, contact the ground/RLD electrode 330 using his/her left-hand index finger or middle finger, contact PPG sensor 320 using his/her right-hand thumb, and contact the ECG right-hand electrode 340 using his/her right-hand index finger or middle finger.

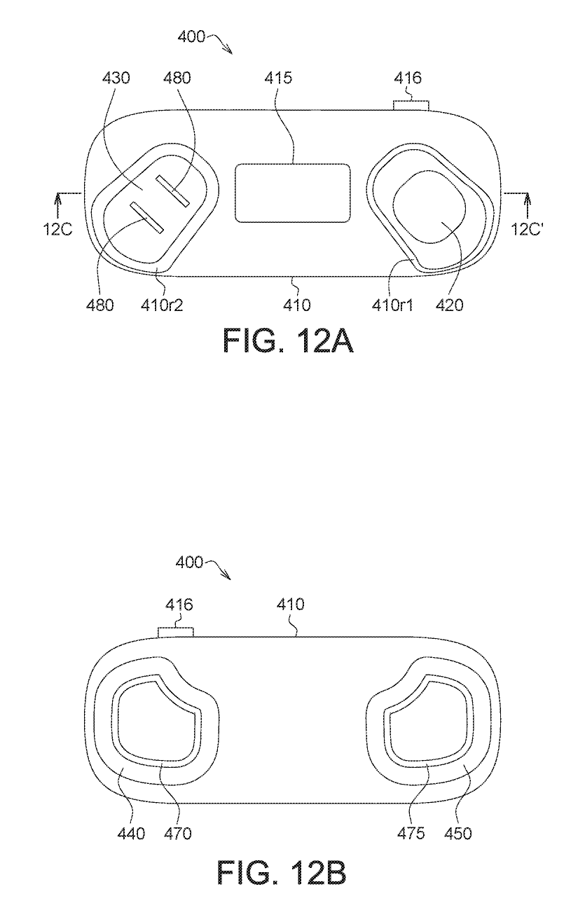

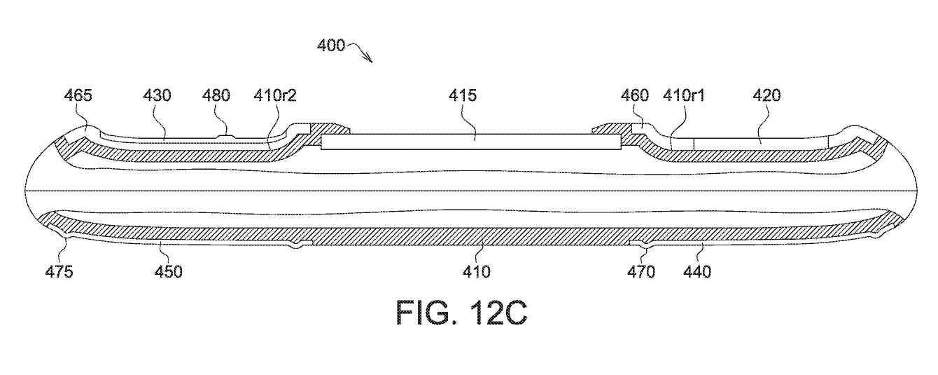

Refer to FIGS. 12A, 12B and 120. FIG. 12A is a front view of a pulse wave velocity meter 400 according to a fourth embodiment of the invention. FIG. 12B is a back view of the pulse wave velocity meter 400 of FIG. 12A. FIG. 120 is a partial cross-sectional view of the pulse wave velocity meter 400 of FIG. 12A viewed along a direction 12C-12C′.

The pulse wave velocity meter 400 includes, for example, a carrier 410 of a casing, a display unit 415, a power key 416, a PPG sensor 420, a PPG sensing unit (not illustrated), a ground/RLD electrode 430, an ECG right-hand electrode 440, an EGG sensing unit (not illustrated), an ECG left-hand electrode 450, a processing unit (not illustrated), a first carrying piece 460, a second carrying piece 465, a first convex ring 470, a second convex ring 475, and at least a protrusion portion 480.

As indicated in FIG. 12B, the carrier 410 has a first recess portion 410r1 and a second recess portion 410r2, wherein the PPG sensor 420 and the ground/RLD electrode 430 respectively are disposed on the first recess portion 410r1 and the second recess portion 410r2. The recess design of the first recess portion 410r1 arid the second recess portion 410r2 can position the finger, such that the finger can easily contact the PPG sensor 420 and the ground/RLD electrode 430.

Furthermore, the PPG sensor 420 and the first carrying piece 460 can be pre-assembled as a first pre-assembled piece first and then together are assembled inside the first recess portion 410r1 of the carrier 410. Similarly, the ground/RLD electrode 430 arid the second carrying piece 465 can be pre-assembled as a second pre-assembled piece first and then together are assembled inside the second recess portion 410r2 of the carrier 410. Thus, the assembling process of the pulse wave velocity meter 400 can be simplified and/or the assembling work of the pulse wave velocity meter 400 can be reduced. In another embodiment, the PPG sensor 420 can be directly disposed inside the first recess portion 410r1 and/or the ground/RLD electrode 430 can be directly disposed inside the second recess portion 410r2.

As indicated in FIG. 12B and 120, the first convex ring 470 is disposed on the ECG right-hand electrode 440. The second convex ring 475 is disposed on the ECG left-hand electrode 450. The protrusion design of the first convex ring 470 and the second convex ring 475 provides the user with a tactile feel at the finger. In an embodiment, the enclosed area of the first convex ring 470 is substantially equivalent to the front-end area of the user's right-hand index finger or middle finger, and/or the enclosed area of the second convex ring 475 is substantially equivalent to the front-end area of the user's left-hand index finger or middle finger. The convex ring guides the user's left-hand/right-hand index finger or middle finger to contact the electrode contact region enclosed by the convex ring and further restrict the finger such that the finger will not easily side to the outside of the said region. Thus, the finger can stably contact the electrode and the contact area between the finger and the electrode can be maximized.

As indicated in FIG. 12A and 120, the protrusion portion 480 can be disposed on the ground/RLD electrode 430 or integrally formed in one piece with the ground/RLD electrode 430 to provide the user with a tactile feel at the finger. Furthermore, the protrusion portion 480 also prevents the finger from sliding outside the ground/RLD electrode 430 easily.

To summarize, since the PPG sensor, the ground/RLD electrode, the ECG right-hand electrode and the ECG left-hand electrode of the embodiments of the invention can be disposed at a plurality of separation regions of the carrier, the PPG sensor, the ground/RLD electrode, the ECG right-hand electrode and/or the ECG left-hand electrode can respectively provide a large and sufficient contact region to improve their contact quality with the human body and increase the accuracy of the measured physiological information.

Besides, as long as the ECG signal and the PPG signal can be obtained, the embodiments of the invention do not restrict the position of the PPG sensor, the ground/RLD electrode, the ECG right-hand electrode and/or the ECG left-hand electrode on the carrier. Moreover, the said carrier can be realized by any suitable component (a component, such as a handle that can contact human body) of a device, wherein the device can be realized by a keypad, desk, a chair, a toilet, a bathroom, an appliance, a transportation (such as vehicle, motor cycle or bicycle) in which the pulse wave velocity meter can be disposed or installed.

The positions of the PPG sensor, the ground/RLD electrode, the EGG right-hand electrode and the ECG left-hand electrode of the embodiments of the invention can be changed according to the shape of the carrier and the user's operating and gripping habits. For example, when the carrier is a mobile phone casing, the PPG sensor, the ground/RLD electrode and the ECG left-hand electrode can be disposed at three separation regions on the rear surface of the mobile phone casing for the user, who is used to hold the mobile phone with his/her left hand, to contact the PPG sensor, the ground/RLD electrode and the ECG left-hand electrode using his/her middle finger, index finger and ring finger respectively. Or, the PPG sensor, the ground/RLD electrode and the ECG left-hand electrode can be disposed on the same lateral surface of the mobile phone casing (for example, the right lateral surface of the mobile phone casing) for the user to contact the PPG sensor, the ground/RLD electrode and the ECG left-hand electrode disposed on the right lateral surface of the mobile phone casing using his/her left-hand middle finger, index finger and ring finger respectively; the ECG right-hand electrode can be disposed on the front surface of the mobile phone casing for the right-handed user to contact the ECG right-hand electrode using his/her right-hand index finger or middle finger. It should be noted that the PPG sensor, the ground/RLD electrode, the ECG right-hand electrode and the ECG left-hand electrode can be adjacent to a long side of the mobile phone casing to match the gripping habit of operating the mobile phone in a vertical manner. Moreover, the PPG sensor, the ground/RLD electrode, the ECG right-hand electrode and the ECG left-hand electrode can also be adjacent to a short side of the mobile phone casing to match the user's gripping habit of operating the mobile phone in a horizontal manner.

When the carrier is a steering wheel, the PPG sensor, the ground/RLD electrode, the ECG right-hand electrode and the ECG left-hand electrode can be disposed on the same side of the steering wheel facing the user. The ECG right-hand electrode and one of the PPG sensor and the ground/RLD electrode can be disposed at the right side of the steering wheel, and the ECG left-hand electrode and the other one of the PPG sensor and the ground/RLD electrode can be disposed at the left side of the steering wheel to match the user's gripping habit of holding the steering wheel using his/her fingers. Or, the ECG right-hand electrode and one of the PPG sensor and the ground/RLD electrode can be disposed on two opposite surfaces at the right side of the steering wheel respectively, and the ECG left-hand electrode and the other one of the PPG sensor and the ground/RLD electrode can be disposed on two opposite surfaces at the left side of the steering wheel respectively to match the user's gripping habit of contacting two opposite surfaces of the steering wheel using several fingers.

While the invention has been described by way of example and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A pulse wave velocity meter, comprising:
a carrier;
a photo-plethysmography (PPG) sensor;
an electrocardiography (ECG) right-hand electrode;
an ECG left-hand electrode;
a PPG sensing unit electrically connected to the PPG sensor;
an ECG sensing unit electrically connected to the ECG right-hand electrode and the ECG left-hand electrode; and
a processing unit electrically connected to the PPG sensing unit and the ECG sensing unit;
wherein the PPG sensor, the ECG right-hand electrode and the ECG left-hand electrode respectively are disposed at a plurality of separation regions of the carrier.

2. The pulse wave velocity meter according to claim 1, further comprising:
a ground/right leg drive (RLD) electrode;
wherein the PPG sensor, the ECG right-hand electrode, the ground/RLD electrode and the ECG left-hand electrode respectively are disposed at the separation regions of the carrier.

3. The pulse wave velocity meter according to claim 2, wherein the carrier comprises a front surface and a rear surface disposed oppositely, the PPG sensor and the ground/RLD electrode are disposed at two separation regions on the front surface, the ECG right-hand electrode and the ECG left-hand electrode are disposed at two separation regions on the rear surface, the PPG sensor is opposite to one of the ECG right-hand electrode and the ECG left-hand electrode, and the ground/RLD electrode is opposite to the other one of the ECG right-hand electrode and the ECG left-hand electrode.

4. The pulse wave velocity meter according to claim 2, wherein the pulse wave velocity meter is a communication device; the carrier is at least a portion of a casing of the communication device; the casing comprises a front surface and a rear surface disposed oppositely; the PPG sensor is disposed on the front surface; the ground/RLD electrode, the ECG right-hand electrode and the ECG left-hand electrode are disposed on the rear surface; the ECG right-hand electrode and the ground/RLD electrode are adjacent to an edge of the casing; the ECG left-hand electrode is adjacent to another edge of the casing.

5. The pulse wave velocity meter according to claim 2, wherein the pulse wave velocity meter is a communication device; the carrier is at least a portion of a casing of the communication device; the casing comprises a front surface and a rear surface disposed oppositely; the PPG sensor, the ground/RLD electrode and the ECG left-hand electrode are disposed on the rear surface; the ECG right-hand electrode is disposed on the front surface.

6. The pulse wave velocity meter according to claim 2, wherein the pulse wave velocity meter is a communication device; the carrier is at least a portion of a casing of the communication device; the casing comprises a front surface and a lateral surface; the ground/RLD electrode and the ECG left-hand electrode are disposed on the lateral surface; the PPG sensor and the ECG right-hand electrode are disposed on the front surface.

7. The pulse wave velocity meter according to claim 2, wherein the pulse wave velocity meter is a steering wheel of a vehicle; the carrier is a handle of the steering wheel; the handle comprises a front surface; the ECG left-hand electrode and the ground/RLD electrode are disposed on a left side of the front surface; the PPG sensor and the ECG right-hand electrode are disposed on a right side of the front surface.

8. The pulse wave velocity meter according to claim 2, wherein the pulse wave velocity meter is a steering wheel of a vehicle; the carrier is a handle of the steering wheel; the handle comprises a rear surface; the PPG sensor and the ECG right-hand electrode are disposed on a right side of the rear surface; the ECG left-hand electrode and the ground/RLD electrode are disposed on a left side of the rear surface.

9. The pulse wave velocity meter according to claim 2, wherein the pulse wave velocity meter is a steering wheel of a vehicle, the carrier is a handle of the steering wheel; the handle comprises a front surface and a rear surface; the PPG sensor is disposed on a right side of the front surface; the ECG right-hand electrode is disposed on a right side of the rear surface; the ECG left-hand electrode is disposed on a left side of the front surface; the ground/RLD electrode is disposed on a left side of the rear surface.

10. The pulse wave velocity meter according to claim 2, wherein the carrier has a second recess portion within which the ground/RLD electrode is disposed.

11. The pulse wave velocity meter according to claim 2, further comprising:
a protrusion portion disposed on the ground/RLD electrode.

12. The pulse wave velocity meter according to claim 1, wherein the carrier has a first recess portion within which the PPG sensor is disposed.

13. The pulse wave velocity meter according to claim 1, further comprising:
a first convex ring disposed on the ECG right-hand electrode.

14. The pulse wave velocity meter according to claim 1, further comprising:
a second convex ring disposed on the ECG left-hand electrode.

15. The pulse wave velocity meter according to claim 1, wherein the carrier is a casing of an electronic device.

16. The pulse wave velocity meter according to claim 1, wherein the carrier is a handle of a steering wheel.

17. The pulse wave velocity meter according to claim 1, wherein the pulse wave velocity meter is a keypad, a communication device or a steering wheel.
